(19) United States
(12) Patent Application Publication
(10) Pub. No.: US 2019/0076442 A1
(43) Pub. Date: Mar. 14, 2019

(54) PRESERVATIVE FREE PHARMACEUTICAL COMPOSITION FOR OPHTHALMIC ADMINISTRATION CONTAINING BIMATOPROST AND TIMOLOL

(71) Applicant: PHARMATHEN S.A., PALLINI-ATTIKIS (GR)

(72) Inventor: EVANGELOS KARAVAS, PALLINI ATTIKIS (GR)

(73) Assignee: PHARMATHEN S.A., PALLINI-ATTIKIS (GR)

(21) Appl. No.: 16/087,718

(22) PCT Filed: Mar. 28, 2017
(86) PCT No.: PCT/EP2017/025069

§ 371 Date: Sep. 24, 2018

(30) Foreign Application Priority Data
Apr. 1, 2016 (GR) 20160100135

(51) Int. Cl. A61K 31/5575; A61K 9/00; A61K 9/08; A61K 31/5377; A61K 47/12; A61K 47/02

(52) U.S. Cl. CPC A61K 31/5575; A61K 9/0048; A61K 47/02; A61K 31/5377; A61K 47/12; A61K 9/08

(57) ABSTRACT

The present invention relates to a preservative free ophthalmic pharmaceutical formulation for topical administration containing a therapeutically effective quantity of Bimatoprost or ophthalmological acceptable salts thereof and a therapeutically effective quantity of Timolol or ophthalmological acceptable salts thereof to be used for the treatment of ocular hypertension and glaucoma.

### TECHNICAL FIELD OF THE INVENTION

The present invention relates to a preservative free ophthalmic formulation for topical administration containing a therapeutically effective quantity of a prostaglandin analogue such as Bimatoprost or ophthalmological acceptable salt thereof and a beta-adrenergic receptor antagonist such as Timolol or ophthalmological acceptable salt thereof to be used for the treatment of ocular hypertension and glaucoma. Such preservative-free formulation is packed in container that ensures physical and chemical stability of the product.

### BACKGROUND OF THE INVENTION

Glaucoma is a group of eye disorders traditionally characterized by progressive damage to the eye, at least partly due to elevated intraocular pressure (IOP). It is the leading cause of irreversible blindness in the world and the second leading cause of vision loss after cataract, which is reversible surgically.

Eyes with glaucoma develop progressive peripheral visual field loss followed by central field loss, in a characteristic pattern, usually but not always in the presence of elevated IOP. There are no anatomic factors that identify eyes that are at risk. Visual field loss cannot be recovered once it has occurred.

Prostaglandin analogues are the most recent pharmacological group to treat topically open angle glaucoma. Instead of decreasing the production of aqueous humour produced by the ciliary body, as beta adrenergic blockers and carbonic anhydrase inhibitors do, these products lower the IOP by means of increasing the aqueous humour outflow through the uveoscleral pathway.

Bimatoprost is a synthetic analog of prostaglandin F₂α ethanolamide (prostamide F₂α), and shares a pharmacological profile consistent with that of the prostamides. Like prostaglandin F₂α carboxylic acid, Bimatoprost potently lowers intraocular pressure with open-angle glaucoma or ocular hypertension. It is a white crystalline, hydroscopic powder with a molecular weight of 415.57 which is slightly soluble in water and very soluble in ethyl alcohol.

In a large proportion of patients with glaucoma or ocular hypertension prostaglandins by themselves do not produce enough pressure reduction to reach the desired target. As a result, many such patients require more than one medication. Beta blockers such as Timolol are usually used as add-on therapy for patients who are already on a prostaglandin therapy. Topical beta-blockers reduce the intraocular pressure (IOP) by blockade of sympathetic nerve endings in the ciliary epithelium causing a fall in aqueous humour production. Timolol maleate is a white to almost white, odorless powder with a molecular weight of 432.5 and is soluble in water, ethanol and methanol; sparingly soluble in chloroform and propylene glycol and insoluble in ether and in cyclohexane.

U.S. Pat. Nos. 4,195,085 and 4,861,760 describe the use of Timolol as an ophthalmic drug.

WO 2012/163827 A discloses an aqueous ophthalmic preparation comprising a PGF2a analogue and at least one polyvinyl alcohol, whereby the preparation is essentially preservative-free.

It is known that combinations of prostaglandin analogues with beta adrenergic receptor antagonists (beta blockers) and especially those already in use for ophthalmological applications such as Timolol can increase the efficacy of the preparation; however, there still remains a need for an effective and safe topical ophthalmic pharmaceutical composition containing Bimatoprost and Timolol which has increased stability and fewer side effects. In particular, there is a need for a combined Bimatoprost-Timolol formulation that is free from preservatives to be provided in a multiple use container and provide efficient dosing of the solution to the patient, without wastage.

### SUMMARY OF THE INVENTION

An object of the present invention is to provide a stable preservative free ophthalmic formulation for topical administration containing a β-adrenergic receptor antagonist and in particular Timolol maleate and a prostaglandin analogue and in particular Bimatoprost or ophthalmologically acceptable salts thereof to be used for the treatment of ocular hypertension and glaucoma, which overcomes the deficiencies of the prior art.

Moreover, an aspect of the present invention is to provide a preservative free ophthalmic formulation for topical administration containing Timolol maleate and Bimatoprost or ophthalmologically acceptable salts thereof, which is bioavailable and effective with sufficient self-life.

Another aspect of the present invention is to provide a method for the preparation of a stable preservative free ophthalmic formulation for intravitreal administration comprising Timolol maleate and Bimatoprost or ophthalmologically acceptable salts thereof, as the active ingredients, permitting enhanced release of the active medicaments that can be used for the treatment of ocular hypertension and glaucoma with improved pharmacotechnical characteristics of the composition.

It is an object of the present invention to provide an efficient ophthalmic product that contains no antimicrobial preservatives and is as effective in terms of therapy as products available with preservatives.

A further approach of the present invention is to provide ophthalmic solutions that are easily administrable in drop form.

In accordance with the above objects of the present invention, an ophthalmic, preservative-free pharmaceutical formulation is provided comprising Bimatoprost or ophthalmologically acceptable salts thereof and Timolol maleate as active ingredients, a tonicity agent and one or more buffering agents.

According to another embodiment of the present invention, a process for the preparation of a preservative-free pharmaceutical formulation for ophthalmic administration containing Bimatoprost or ophthalmologically acceptable salts thereof and Timolol maleate is provided, comprising the following stages:
Adding the tonicity agent into water for injection and dissolving;
Adding to the solution formed the buffering agent under stirring until dissolution;
Adding to the solution formed a second buffering agent under stirring until dissolution;
Adding Timolol maleate into solution of previous step under stirring until dissolution;
Adding Bimatoprost and stirring the solution until complete dissolution;
Adjusting the pH of the solution to 7.30 by adding either sodium hydroxide or hydrochloric acid;
Adjusting final solution volume using water for injections and checking again pH of solution;
Adjusting again the pH of the solution to 7.30, if necessary, by adding either sodium hydroxide or hydrochloric acid.

Other objects and advantages of the present invention will become apparent to those skilled in the art in view of the following detailed description.

### DETAILED DESCRIPTION OF THE INVENTION

For the purposes of the present invention, a pharmaceutical composition comprising an active ingredient is considered to be “stable” if said ingredient degrades less or more slowly than it does on its own and/or in known pharmaceutical compositions.

Ocular administration of drugs is primarily associated with the need to treat ophthalmic diseases. Eye is the most easily accessible site for topical administration of a medication. Ophthalmic preparations are sterile products essentially free from foreign particles, suitably compounded and packaged for instillation into the eye. They are easily administered by the nurse or the patient himself, they have quick absorption and effect, less visual and systemic side effects, increased shelf life and better patient compliance.

Antimicrobial preservatives are added to aqueous preparations that are required to be sterile, such as in ophthalmic solutions. The use of preservatives in topical ophthalmic treatments is ubiquitous for any product that is to be used more than once by the patient as they prevent any microbes that may enter into the product after its first use from allowing those microbes to grow and infect the patient on a later use of the product. Although providing effective biocidal properties with well tolerated short-term use at low concentrations, preservatives can cause serious inflammatory effects on the eye with long-term use in chronic conditions, such as glaucoma or potentially ocular allergies.

Antimicrobial preservatives are not found in single use vials of ophthalmic solutions since they are manufactured aseptically or are sterilised and the products are used once and the dispenser is thrown away.

Preservative-free single dose containers most often are presented as blow-fill-seal (BFS) containers. The user takes the plastic vial and tears or cuts the plastic tip, inverts the vial and squeezes the ophthalmic liquid into the eye. Disadvantages of these systems are linked to the quite complicated filling technology, the need to overfill and amount of material needed for each dose. With an average drop size of ˜35 μl and the standard commercial volume of 400-500 μl, five times the required drug quantity ends up being discarded in case of single dose containers. Additionally, a big amount of packaging material is required associated with high manufacturing costs. A further disadvantage is that, despite numerous technical improvements were made by some manufacturers, the edges around the tip of the opened dropper of disposable, single-dose container are still very sharp, which may cause an accident to the patients eye.

As the use of preservative containing eye drops has been implicated in the development or worsening of ocular surface disease, there is a tendency to limit their use by reducing their concentration as much as possible in eye drops. The present invention provides completely preservative-free ophthalmic formulations. Such formulations are packed in containers that enable to deliver preservative-free formulations while providing shelf life similar to traditional formulations. The containers of the present invention ensure that medication is kept germ-free even after multiple uses.

Patient compliance is greatly increased as the pumps of the present invention permit them to use preservative-free eye drops without worrying about the potential side effects caused by some preservatives and the related short- and long-term consequences, such as pain or discomfort, foreign body sensation, stinging or burning, dry eye sensation, ocular surface breakdown.

We have found that the design of the tip of the container produce a highly accurate drop size with low variability of drop volume between each drop dispensed.

Therefore, we present as a feature of the present invention a multi-use ophthalmic product comprising a container with an integral bacterial protection system and which has a dispensing tip, wherein the ratio of the inner to the outer diameter of the dispensing tip is from 1:1 to 1:6, and the container having an ophthalmic composition that is dispensed from the tip into the eye of a patient wherein the ophthalmic composition is a preservative-free aqueous solution and contains pharmaceutically acceptable excipients.

Tonicity refers to the osmotic pressure exerted by salts in aqueous solution. An ophthalmic solution is isotonic with another solution when the magnitudes of the colligative properties of the solutions are equal. An ophthalmic solution is considered isotonic when its tonicity is equal to that of 0.9% sodium chloride solution (290 mOsm). This requires that a certain tonicity agent be added so that the total osmotic pressure is the same as the body fluid.

Sodium chloride, mannitol, dextrose, glycerine, potassium chloride are typical tonicity agents. Preferably, sodium chloride is used in the present invention as tonicity agent.

The aqueous formulation according to the present invention comprises sodium chloride in a range from 0.61% to 0.81% (w/v), preferably 0.71% (w/v).

Preferred compositions are prepared using a buffering system that maintains the composition at a pH of about 7 to a pH of about 7.8, preferably 7.1-7.5, and most preferably 7.3.

Suitable buffering agents include, but are not limited to, dibasic sodium phosphate heptahydrate, citric acid monohydrate, monobasic sodium phosphate, disodium phosphate dodecahydrate, hydrochloric acid, sodium hydroxide, sodium hydrogen carbonate. Preferably, dibasic sodium phosphate heptahydrate and citric acid monohydrate are used in the present invention as buffering agents.

The aqueous formulation according to the present invention comprises buffering agent in a range from 0.2% to 0.3% (w/v), preferably 0.28% (w/v).

### EXAMPLES

### Example 1

Preservative-free ophthalmic compositions comprising Bimatoprost and Timolol maleate according to the present invention are illustrated in Table 1 below:

TABLE 1
Compositions 1 to 3

Compositions

| | | |
|---|---|---|
| 1 | 2 | 3 |

mg/mL

| | | | |
|---|---|---|---|
| Bimatoprost | 0.300 | 0.300 | 0.300 |
| Timolol Maleate | 6.830 | 6.830 | 6.830 |
| (equivalent to Timolol) | 5.000 | 5.000 | 5.000 |
| Sodium chloride | 6.700 | 7.500 | 7.100 |
| Sodium phosphate dibasic heptahydrate | 3.680 | 2.680 | 2.680 |
| Citric Acid monohydrate | 0.140 | 0.140 | 0.140 |

| | |
|---|---|
| NaOH/HCl | q.s. to pH 7.3 |
| Total solution volume (ml) | 1.00 |

A range of alternative compositions were prepared alternating either the sodium phosphate dibasic heptahydrate content or the sodium chloride content.

The manufacturing process followed in all compositions is described below:
Adding sodium chloride into water for injection and dissolving;
Adding to the solution formed sodium phosphate dibasic heptahydrate under stirring until dissolution;
Adding citric acid monohydrate into solution of previous step under stirring until dissolution;
Adding Timolol maleate and stirring the solution until complete dissolution;
Adding Bimatoprost and stirring the solution until complete dissolution;
Adjusting the pH of the solution to 7.30 by adding either sodium hydroxide or hydrochloric acid;
Adjusting final solution volume using water for injections and checking again pH of solution;
Adjusting again the pH of the solution to 7.30, if necessary, by adding either sodium hydroxide or hydrochloric acid.

The physicochemical properties and assay of Compositions 1-3 are presented in table 2 below:

TABLE 2
Physicochemical properties and Assay of Compositions 1-3

| | Composition | | |
|---|---|---|---|
| | 1 | 2 | 3 |
| pH | 6.03 | 5.78 | 5.75 |
| pH adjustment (NaOH/HCl) | 7.30 | 7.30 | 7.30 |
| Osmolality (mOsmol/kg) | 285 | 302 | 290 |
| Surface tension (mN/m) | 54.83-55.63 | 54.92-55.36 | 55.21-55.59 |
| Viscosity (cP) - 100 rpm, | 1.34 | 1.34 | 1.36 |
| spindle 00 | | | |
| Specific gravity | 1.009 | 1.009 | 1.009 |
| Appearance | clear, colorless to slightly yellow solution | | |
| Assay Bimatoprost | 99.3% | 98.9% | 100.3% |
| Assay Timolol | 98.7% | 99.5% | 100.6% |

The preferred composition of the present invention is Composition 3 as the physicochemical results of Composition 3 were acceptable and within specifications.

In order to ensure that the filter used during the manufacturing process does not retain the drug substances Bimatoprost and Timolol maleate and does not cause impurities to the final product, a filter study was performed. The procedure simulated the production filtration, by using different filter membranes. Samples of the solution of Composition 3 before and after filtration were collected and were analyzed under assay and impurities method determination. Totally, four membrane materials were tested-PVDF, PTFE, PES and NYLON.

The % Assay of API after filtration should be between ±2% of Assay before filtration.

The % difference in Total impurities after filtration should be not more than 5% compared to the Total impurities before filtration.

TABLE 3
Results of filter selection study for Bimatoprost 0.3
mg/mL/Timolol maleate 5 mg/mL, eye drops solution.
Before filtration
Assay

| Sample | % Assay Bimatoprost | % Assay Timolol |
|---|---|---|
| Average before | 98.9 | 98.7 |

Impurities

| SPECIFICATIONS (LIMIT) | % Impurities |
|---|---|
| Total Bimatoprost (NMT 5.0%) | 0.32 |
| Total Timolol (NMT 3.0%) | 0.01 |

After filtration
Assay

| | % Assay | | | |
|---|---|---|---|---|
| | Filter 1 | Filter 2 | Filter 3 | Filter 4 |
| Sample | PVDF | PES | PTFE | NYLON |
| Bimatoprost Assay % | 98.5 | 98.6 | 98.6 | 88.8 |
| Timolol Assay % | 98.1 | 98.6 | 98.6 | 98.5 |

Impurities

| | % Impurities | | | |
|---|---|---|---|---|
| | Filter 1 | Filter 2 | Filter 3 | Filter 4 |
| SPECIFICATIONS (LIMIT) | PVDF | PES | PTFE | NYLON |
| Total Bimatoprost (NMT 5.0%) | 0.34 | 0.37 | 0.36 | 0.60 |
| Total Timolol (NMT 3.0%) | 0.01 | 0.01 | 0.01 | 0.01 |

From the results of all filters studied, a small absorption of Bimatoprost API is observed on NYLON filters. Additionally, NYLON filters are susceptible to hydrolysis and it has been observed that substances are leached from the filter along the filtration process. For that reason, Nylon membrane was excluded from the current product development.

PVDF filter is selected to be used in the manufacturing process of Bimatoprost/Timolol maleate eye drops solution of the present invention.

After concluding to the PVDF membrane, a more extensive study was performed in order to examine in details the filter-solution compatibility and the filter adsorptive effects.

The first test assures the chemical compatibility between filter and solution and that the membrane is integral after being subjected in contact with the product for 24 hours. The second test evaluates the filter adsorptive properties on the API. It is critical that filters are selected to minimize adsorption and loss of product components.

The PVDF membrane was immersed in Bimatoprost/Timolol PF 0.3 mg/mL+5 mg/mL eye drops solution and it was kept at 25° C. for 24 hours. The solution was protected from light. Samples were taken at zero time and at 4 hour intervals and were tested for their assay content and impurity profile. Results are presented in table 4 below:

TABLE 4
Results of filter compatibility study for Bimatoprost
0.3 mg/mL/Timolol maleate 5 mg/mL, eye drops solution.
A. Solution at zero time
Assay

| Sample | % Assay |
|---|---|
| Assay Bimatoprost | 100.7 |
| Assay Timolol | 98.4 |

Impurities

| SPECIFICATIONS (LIMIT) | % Total Impurity |
|---|---|
| Total impurities Bimatoprost (Total NMT 5.0%) | 0.46 |
| Total impurities Timolol (Total NMT 3.0%) | 0.01 |

B. Solution in contact with the filter
Assay

% Assay

| Sample | 4 hrs | 8 hrs | 12 hrs | 24 hrs |
|---|---|---|---|---|
| Assay Bimatoprost | 100.5 | 99.8 | 100.1 | 100.5 |
| Assay Timolol | 98.4 | 97.0 | 97.8 | 99.3 |

Impurities

| | % Impurities | | | |
|---|---|---|---|---|
| SPECIFICATIONS (LIMIT) | 4 hrs | 8 hrs | 12 hrs | 24 hrs |
| Bimatoprost (Total NMT 5.0%) | 0.44 | 0.48 | 0.44 | 0.46 |
| Timolol (Total NMT 3.0%) | 0.01 | 0.01 | 0.01 | 0.01 |

The average % Assay for Bimatoprost API and Timolol API is not decreased when the solution is in intimate contact with the filter for 24 hours, indicating that no adsorption from the filter occurs. Also, it was not observed any significant increment at the impurities, thus the filter is inert for this product and can be safely used for the manufacturing of the current development.

Storage of the final product at zero time, 3 and 6 months under long term (25° C./60% RH), intermediate (30° C./65% RH) and accelerated storage conditions (40° C./75% RH) did not alter significantly the related substances profile conforming to the specification limits. Consequently, the multi-dose PF system of the present invention is indicated for the current development since the related substances of Bimatoprost and Timolol are within the specifications in all storage conditions.

In order to investigate the potential contamination of the tip during use, i.e. by accidently touching the eye, a microbial challenge test has been performed for the optimized formulation. A challenge suspension containing Brevundimonas Diminuta (ATCC 19146) was prepared. The dropper of the multi-dose PF system was actuated by immersing the tip in challenge suspension and left at room temperature in order to simulate in use conditions.

The sterility of the optimized formulation was also checked upon storage in the multi-dose PF container for 6 months at 40° C. The results of these tests are presented in table 5 below.

TABLE 5
Results of sterility tests for Bimatoprost 0.3 mg/mL/Timolol maleate
5 mg/mL, eye drops solution in the multi-dose PF container.

| | | |
|---|---|---|
| Test | Requirements | Result |

Sterility upon storage

| | | |
|---|---|---|
| Product at zero-time | Sterile | Conforms |
| Product after storage for | Sterile | Conforms |
| 6 months at 40° C. | | |

In use sterility test

| | | |
|---|---|---|
| Product at zero-time | Sterile | Conforms |
| Product after in-use test | Sterile | Conforms |

In use sterility challenge test

| | | |
|---|---|---|
| Product at zero-time | Sterile | Conforms |
| Product after challenging | Sterile | Conforms |
| and incubation | | |

It is obvious that the multi-dose PF container meets the sterility requirements for Bimatoprost 0.3 mg/mL/Timolol maleate 5 mg/mL, eye drops solution.

While the present invention has been described with respect to the particular embodiment, it will be apparent to those skilled in the art that various changes and modifications may be made in the invention without departing from the spirit and scope thereof, as defined in the appended claims.

1. A preservative free ophthalmic pharmaceutical composition comprising a therapeutically effective quantity of Bimatoprost or ophthalmologic acceptable salt thereof and a therapeutically effective quantity of Timolol or ophthalmologic acceptable salt thereof.

2. The preservative free ophthalmic pharmaceutical composition according to claim 1, wherein the quantity of Bimatoprost is about 0.03% by weight.

3. The preservative free ophthalmic pharmaceutical composition according to claim 1, wherein the quantity of Timolol is about 0.5% by weight.

4. The preservative free ophthalmic pharmaceutical composition according to claim 1, wherein it further comprises effective amount of one or more buffering agents and a tonicity agent.

5. The ophthalmic pharmaceutical composition according to claim 4, wherein the buffering agent is selected from dibasic sodium phosphate heptahydrate and citric acid monohydrate and the tonicity agent is sodium chloride.

6. The ophthalmic pharmaceutical composition according to claim 4, wherein the amount of buffering agent in the composition is from 0.2% to 0.3% w/v.

7. The ophthalmic pharmaceutical composition according to claim 5, wherein the amount of sodium chloride in the composition is from 0.61% to 0.81% w/v.

8. The ophthalmic pharmaceutical composition according to claim 1, wherein the pH value is between 7.1 and 7.5.

9. The ophthalmic pharmaceutical composition according to claim 1, wherein the composition is sterilized under filtration with hydrophilic modified PVDF membrane.

10. The ophthalmic pharmaceutical composition according to claim 1, wherein the composition is packed in a multi-use container equipped with an integral bacterial protection system.
